(19) United States
(12) Patent Application Publication
(10) Pub. No.: US 2007/0197638 A1
(43) Pub. Date: Aug. 23, 2007

(54) Weight loss induced by reduction in neuropeptide level

(75) Inventors: Thomas M. Loftus, Great Falls, VA (US); Craig A. Townsend, Baltimore, MD (US); Gabrielle Ronnett, Lutherville, MD (US); M. Daniel Lane, Baltimore, MD (US); Francis P. Kuhajda, Lutherville, MD (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: The Johns Hopkins University School of Medicine Licensing and Technology Development, Baltimore, MD (US)

(21) Appl. No.: 11/657,536

(22) Filed: Jan. 25, 2007

Related U.S. Application Data
(63) Continuation of application No. 10/476,513, filed on Oct. 31, 2003, now abandoned.
(60) Provisional application No. 60/208,560, filed on Jun. 2, 2000.
(60) Provisional application No. 60/182,901, filed on Feb. 16, 2000.

(51) Int. Cl. A61K 31/365; C12Q 1/68

(52) U.S. Cl. 514/473; 435/6

(57) ABSTRACT

This invention provides a method for inducing weight loss in an animal by administering to the animal a compound which reduces the expression and/or secretion of neuropeptide Y (NPY). The effect may be accomplished directly, indirectly, or humorally. Preferably, administration of this compound has the effect of increasing malonyl CoA levels in the animal. Compounds administered according to this invention may be inhibitors of fatty acid synthase (FAS), including substituted α-methylene-β-carboxyl-γ-butyrolactones, or inhibitors of malonyl Coenzyme A decarboxylase (MCD). Preferably, the compound is administered in an amount sufficient to reduce the amount and/or duration of expression and/or secretion of NPY to levels at or below those observed for lean animals. In another preferred embodiment, the administration will reduce expression and/or secretion to levels observed for fed or satiated animals; more preferably, administration will reduce the level of NPY below that of fed animals. In a particular embodiment, this invention provides a method for inducing weight loss in an animal by administering a compound which inhibits feeding behavior in the animal. The method is particularly useful for inducing weight loss in animals deficient in expression of the hormone leptin or animals resistant to the action of leptin.

The work leading to this invention was supported in part by Grant Nos. DK0923, DK14575, and DC02979 from the National Institutes of Health and a grant from the Department of the Army. The U.S. Government retains certain rights in this invention.

### BACKGROUND OF THE INVENTION

1. Field of the Invention

This invention is directed to methods of inducing weight loss in an animal. In part, this invention concerns methods for reducing adipocyte mass by controlling the level of neuropeptide Y in the animal.

2. Review of Related Art

Body fat mass is controlled by a complex group of feedback pathways that monitor fat mass and feeding status and regulate feeding and energy utilization. According to the lipostat model originally set forth by Kennedy (Kennedy, G., 1953 “The role of depot fat in the hypothalamic control of food intake in the rat,” Proc. Royal Soc. London (Biol), 140:579-592), peripheral signals from adipose tissue, gut and liver and pancreas act on neurons in the. hypothalamus to modulate energy homeostasis. A number of the regulatory pathways involved have recently been identified.

The best known of the peripheral signals of feeding and adiposity include leptin, insulin, and the gut satiety peptides. Leptin, a cytokine-related hormone produced primarily by adipocytes, is released in proportion to adipose mass. Thus it acts as a signal of adipose mass, both peripherally and in the feeding control centers of the hypothalamus, to inhibit feeding and promote weight loss (Hwang, C., et al., 1997, “Adipocyte differentiation and leptin expression,” Annual Review of Cell & Developmental Biology, 13:231-259). Leptin levels are also elevated by feeding, reflecting feeding status as well as adiposity. Lack of leptin, as observed in the ob/ob mouse (Coleman, D. L., 1978, “Obese and diabetes: Two mutant genes causing diabetes-obesity syndromes in mice,” Diabetologia, 14:141-148) and certain human individuals (Montague, C., et al., 1977, “Congenital leptin deficiency is associated with severe early-onset obesity,” Nature, 387(6636):903-908), leads to profound early-onset obesity. Insulin, produced by pancreatic beta cells is also produced in proportion to adiposity and in response to feeding. While acting to promote energy storage in the periphery, in the hypothalamus insulin acts in a manner similar to leptin, inhibiting feeding and promoting increased energy utilization (Chavez, M., et al., 1996, “Central insulin and macronutrient intake in the rat,” Am J Physiol, 271:R727-731). The gut peptides (e.g. bombesin and cholecystokinin) are released in response to feeding and act as a signal of meal size (Laburthe, M., et al., 1994, “Receptors for gut regulatory peptides,” Baill Clin Endocinol Metab., 8:77-110). Unlike insulin and leptin, which act by a humoral route, these signals are carried to the brain primarily by afferent sensory neurons of the parasympathetic peripheral nervous system, (i.e. the vagus nerves). Other abdominal signals of feeding status are similarly transmitted.

The regulation of feeding and energy utilization in the brain is controlled primarily through integration of feeding signals in the hypothalamus. Two distinct groups of regulatory neurotransmitters/neuropeptides are coordinately counterregulated depending on the energy status of the individual. Under conditions of energy deficit, signalled by such things as low leptin levels, anabolic signals are activated that stimulate feeding and reduce energy utilization while catabolic signals, which inhibit feeding and increase energy utilization are downregulated. Conversely, under conditions of energy surplus, anabolic signals are downregulated: while catabolic signals are upregulated (Loftus, T., 1999, “An Adipocyte-central nervous system regulatory loop in the control of adipose homeostasis,” Sem. Cell. Dev. BioL, 10(1):11-18).

The best known anabolic signal is neuropeptide Y (NPY). This neuropeptide is produced in the hypothalamus in response to fasting (Schwartz, M., et al., 1998, “Effect of fasting and leptin deficiency on hypothalamic neuropeptide Y gene transcription in vivo revealed by expression of a lacZ reporter gene,” Endocrinology, 139(5):2629-2635) and strongly stimulates feeding (O'Shea, D., et al., 1997, “Neuropeptide Y induced feeding in the rat is mediated by a novel receptor,” Endocrinology, 138(1):196-202). Several of the feeding inhibitory catabolic signals include inhibition of NPY signalling among their mechanisms of action. Other anabolic signals include agouti related peptide (AGRP) (Shutter, G. M., et al., 1997, “Hypothalamic expression of ART, a novel gene related to agouti, is up-regulated in obese and diabetic mutant mice, Genes and Development, 11:593-602) which antagonises the α-MSH receptor (see below), melanin concentrating hormone (MCH) (Ludwig, D., et al., 1998, “Melanin-concentrating hormone: a functional melanocortin antagonist in the hypothalamus,” Am. J: Physiol., 274:(E627-633)) and Orexins A, and B (Sakurai, T., et al., 1998, “Orexins and orexin receptors: a family of hypothalamic neuropeptides and G protein-coupled receptors that regulate feeding behavior,” Cell, 92(4):573-585), also known as hypocretins 1 and 2.

Among catabolic signals, the most central is (α-melanocyte stimulating hormone (α-MSH). This peptide is elevated in response to energy surplus and inhibits feeding and promotes catabolic activity. Mice carrying a deletion in the α-MSH MC4 receptor develop obesity (Huszar, D., et al., 1997, “Targeted disruption of the melanocortin-4 receptor results in obesity, Cell, 88(1):131-141). Similarly, mice overexpressing an antagonist of this receptor such as agouti or AGRP also develop late-onset obesity (Graham M., S. J., et al., 1997, “Overexpression of Agrt leads to obesity in transgenic mice,” Nat. Genetics, 17:273-274). Two additional hypothalamic signals, cocaine and amphetamine regulated transcript: (CART) (Lambert, P., 1998, “CART peptides in the central control of feeding and interactions with neuropeptide Y,” Synapse,. 29(4):293-298) and corticotropin releasing hormone (CRH) (Raber et al. 1997), respond to high levels of feeding signals such as leptin and inhibit feeding. Other signals known to inhibit feeding signals in the brain include neurotensin (Sahu, A., 1998, “Evidence suggesting that galanin (GAL), melanin-concentrating hormone (MCH), neurotensin (NT), proopiomelanocortin (POMC) and neuropeptide Y (NPY) are targets of leptin signaling in the hypothalamus,” Endocrinology, 139(2):795-798), glucagon-like peptide (Turton, M., et al., 1996, “A role for glucagon-like peptide-1 in the central regulation of feeding,” Nature, 379(6560):69-72) and serotonin (Currie, P., et al., 1997, “Stimulation of 5-HT(2A/2C) receptors within specific hypothalamic,” Neuroreport, 8(17):3759-3762). Serotonin has been linked to the appetite suppression observed in anorexia and is the target of the recently withdrawn weight-loss therapy, phen fen.

C-75 is a specific inhibitor of fatty acid synthase (FAS) as disclosed in U.S. Pat. No. 5,981,575, incorporated herein by reference. FAS is one of the primary biosynthetic enzymes of fatty acid synthesis in humans and other mammals (Wakil, 1989, “Fatty acid synthase, a proficient multifunctional enzyme,” Biochemistry, 28:4523-4530). Administration of C-75 to BALB/c mice leads to loss of 10-20% of total body weight within a 24 hour period, lasting for several days with total duration depending of dose. Following this period, body weight returns to normal with no obvious long term effect on the animal.

Excess body weight is a major health problem in developed nations, affecting over 50% of the U.S. population (Must, et al., 1999, J. Amer. Med. Assoc., 282:1523), and is increasing both in prevalence and severity. This condition is associated with increased risk of type II diabetes, cardiovascular and cerebrovascular disease among other disorders as well as significantly increased mortality (Must, et al., 1999). The magnitude of this health problem and the recent difficulties with several weight-loss therapies emphasize the need for, a novel approach to weight loss therapy.

### SUMMARY OF THE INVENTION

It is a object of this invention to promote weight loss by inhibiting feeding behavior. This and other objects are met by one or more of the following embodiments.

In one embodiment, this invention provides a method for inducing weight loss in an animal, the method comprising administering to the animal a compound which reduces the expression and/or secretion of neuropeptide Y (NPY) directly or humorally. Preferably, administration of this compound has the effect of increasing malonyl CoA levels in the animal. Compounds administered according to this invention may be inhibitors of fatty acid synthase (FAS), including substituted α-methylene-β-carboxyl-γ-butyrolactones, or inhibitors of malonyl Coenzyme A decarboxylase (MCD). Preferably, the compound is administered in an amount sufficient to reduce the amount and/or duration of expression and/or secretion of NPY to levels at or below those observed for lean animals. In another preferred embodiment, the administration will reduce expression and/or secretion to levels observed for fed or satiated animals; more preferably, administration will reduce the level of NPY below that of fed animals.

In a particular embodiment, this invention provides a method for inducing weight loss in an animal by administering a compound which inhibits feeding behavior in the animal. The method is particularly useful for inducing weight loss in animals deficient in expression of the hormone leptin or animals resistant to the action of leptin.

In another embodiment, this invention provides a screening method for identifying genes whose expression is associated with control of weight loss. This method comprises comparing mRNA species expressed in tissues of an animal treated with a weight loss agent to mRNA species expressed in corresponding tissues of control animals. Preferably, the treated animal is treated with an FAS inhibitor, more preferably the FAS inhibitor is an substituted α-methylene-β-carboxyl-γ-butyrolactone, such as C-75. In a preferred embodiment of this method, the expressed mRNA is mRNA expressed in hypothalamic tissues. By comparing mRNA expression between treated and control animals, mRNA species associated with genes whose expression is either up-regulated or down-regulated by the weight loss agent ma be identified.

A combination of anabolic and catabolic signals control the body's perception of feeding status. By altering the control of these signals, it is possible to create the perception of the fed or fasted state regardless of the dietary status of the individual. By inhibiting the anabolic signals and activating the catabolic signals, it is possible to induce weight loss, not only through the suppression of feeding, but also by maintaining a normal rate of metabolism, in contrast to the lowered metabolic rate that normally accompanies weight loss.

It has been discovered that FAS inhibitors, such as the α-methylene-β-carboxy-γ-butyrolactone C-75, induce weight loss primarily by an inhibition of feeding (see Example 1). At a sufficient dose, C-75 will completely block all feeding behavior. Furthermore, the observed weight loss can be largely reversed by forced feeding of drug treated animals. C-75 inhibited expression of the prophagic signal neuropeptide Y in the hypothalamus and acted in a leptin-independent. manner that appears to be mediated by malonyl-CoA

There may also be an effect on metabolic rate. C-75 treatment leads to greater weight loss than total food restriction alone (see Example 2). The normal response to fasting in mammals is to reduce the metabolic rate in order to conserve energy. Agents that signal a fed state to the body not only inhibit feeding, but also maintain an elevated metabolic rate, resulting in greater weight loss than lack of feeding alone. This elevation of metabolic rate may also account for the incomplete reversal of weight loss by feeding alone.

### BRIEF DESCRIPTION OF THE FIGURES

FIG. 1.1 shows the structures for cerulenin and C-75 (Panel A), as well as fatty acid synthesis (Panel B) and hepatic malonyl-CoA level (Panel C) in control and C-75-treated mice.

FIG. 1.2 shows body weight (Panel A) and food intake (Panel B) for mice treated with C-75 or RPMI vehicle.

FIG. 2 depicts mice with or without C-75 treatment compared to fasting mice. Panels show (A) body weight and (B) neuropeptide Y mRNA. FIG. 2C shows reversal of the feeding-inhibitory effects of C-75 by intracerebroventricular administration of NPY, thus demonstrating that the animals are capable of responding to NPY if they were not prevented from making it. Panel D shows the effect of C-75 on feeding interval.

FIG. 3 shows leptin independence of the C-75 effects in ob/ob (leptin. deficient) mice. Various panels show (A) leptin levels, (B) weight change, (C) representative individuals, and (D) photomicrographs of control and treated liver.

FIG. 4 shows the effect of C-75 on serum glucose in (A) ob/ob mice and (B) wildtype mice.

FIG. 5 (A) shows a model of feeding regulation by inhibitors of FAS via malonyl-CoA. Panel B shows the interaction of inhibitors of ACC and FAS. Panel C shows the effect of intracerebroventricular injection of C-75.

### DETAILED DESCRIPTION OF THE EMBODIMENTS

The role of metabolism in controlling feeding is well established. The infusion of physiological fuels such as glucose (Grossman, et al., 1997, Physiol. Behav., 61:169) or fatty acids (Scharrer, 1999, Nutrition, 15:704) has long been known to inhibit feeding. Furthermore, anti-metabolites of these substrates also lead to stimulation of feeding, as observed following ICV administration of 2-deoxyglucose, a non-metabolizable glucose analog (Grossman, et al., 1997). There is also a precedent for the control of feeding by alteration of lipid metabolism, as inhibitors of fatty acid oxidation in the liver lead to increased feeding (Scharrer, 1999). However, inhibition of FAS differs from these other metabolic feeding control mechanisms in that it induces a feeding-inhibitory signal in the absence of an added physiological fuel.

A linkage between feeding-inhibition and fatty acid synthesis is consistent with the fact that fatty acid synthesis occurs only during energy surplus, when excess physiological fuels are being channeled into energy storage. A well-characterized regulatory mechanism has been described through which fatty acid synthesis regulates fatty acid oxidation (Rasmussen, et al., 1999, Ann. Rev. Nutri., 19:463). In this paradigm, malonyl-CoA, a substrate for FAS, is elevated during fatty acid synthesis and inhibits carnitine palmitoyl transferase-mediated uptake of fatty acids into the mitochondrion. This regulatory mechanism prevents fatty acid synthesis and oxidation of fatty acids from occurring simultaneously. Elevated malonyl-CoA associated with fatty acid synthesis (See U.S. Patent Application No. 60/164,765 “Modulation of Cellular Malonyl-CoA Levels as a Means to Selectively Kill Cancer Cells,” incorporated herein by reference) may similarly be linked to feeding control.

It is unlikely that inhibition of fatty acid synthesis per se leads to feeding inhibition. Previous studies involving administration of TOFA (Halvorson, et al., 1984, Lipids, 19:851), an inhibitor of acetyl CoA carboxylase (ACC), the enzyme preceding FAS in the fatty acid synthetic pathway, led to inhibition of fatty acid synthesis, but did not inhibit feeding (Malewiak, et al., 1985, Metabolism, 34:604). TOFA administration would be expected to block malonyl-CoA production and thus would not be expected to inhibit feeding. In contrast, inhibition of FAS by C-75 leads to dramatic elevation of malonyl-CoA levels (see U.S. patent application No. 60/164,765) that may mimic active fatty acid synthesis and thus, the fed state.

Fatty acid synthesis regulates fatty acid oxidation via rising malonyl-CoA levels during fatty acid synthesis, which results in inhibition of carnitine palmitoyl transferase-1-mediated uptake of fatty acids into the mitochondrion. This results in elevation of cytoplasmic long-chain fatty acyl-CoA's and diacylglycerol, molecules that may play a signaling role, leading to the proposal that malonyl-CoA levels act as a signal of the availability of physiological fuels.

The mechanism through which FAS inhibition leads to suppression of NPY signaling is unlikely to be related to the mechanism of feeding control by fatty acid oxidation, as feeding control by fatty acid oxidation is mediated by parasympathetic sensory neurons in a process independent of hypothalamic control (Scharrer, 1999). Such sensory neurons have also been reported to play a role in signaling by gut satiety peptides and by leptin (Niijima 1998, J. Auton. Nerv. Syst., 73:19). The gut peptides are also unlikely mediators of this effect as they typically lead to decreased meal size, but not to an overall decrease in food intake or body weight (West, et al., 1984, Am. J. Physiol., 246:R776). However, mediation of FAS effects on feeding by afferent peripheral neurons remains a possible mechanism of such a feeding signal, as these neurons innervate the major sites of fatty acid synthesis, notably the liver and adipose tissue.

Substantial expression of FAS, ACC, and MCD have been observed in selective neuronal populations within the brain such as: the arcuate nucleus, cerebellum, brainstem, hippocampus, and cortex. It is unclear what role these enzymes play as neurons are not thought to carry out significant levels of fatty acid synthesis; however, these neurons possess the machinery to undergo elevation of malonyl-CoA in the presence of C-75 or cerulenin. Studies with [5-³H]-C-75 indicate that the drug enters the brain. Thus, these inhibitors may act directly on the brain to control the feeding centers, either in neurons of the arcuate nucleus itself or in neurons that act on them. The efficacy of C-75 in animals depleted of serotonin by pretreatment with the tryptophan hydroxylase inhibitor, para-Chloro-phenylalanine (Yang, et al., 1995, Am. J. Physiol., 268:E389), argues against that neurotransmitter as a mediator of this effect.

Alternatively, the signal from the FAS target tissue to the hypothalamus may be mediated by a humoral signal. This FAS-associated signal appears to be independent of the systemic release of the known feeding inhibitory hormones leptin and insulin, and the pro-inflammatory cytokines tumor necrosis factor-α and interleukin-1β. Nor is it reversed by administration of dexamethasone, a synthetic glucocorticoid. Necropsy and histological analysis of all major organs in treated mice revealed no adverse pathology and plasma alanine aminotransferase activity was unchanged. In addition, C-75-induced weight loss was observed in mice lacking IL-1r and TNFαrla receptors suggesting that the weight loss is not mediated by an inflammatory response.

In addition to NPY, several other regulatory molecules combine in the hypothalamus to control feeding (Loftus, 1999). The expression of these signals are coordinately regulated either in concert with NPY (e.g. agouti-related peptide) or in opposition to NPY (e.g. α-melanocyte stimulating hormone), depending on feeding status and adiposity. Control of NPY by C-75 may also extend to these co-regulated molecules.

One role proposed for malonyl-CoA is the mediation of nutrient-stimulated insulin secretion in the beta cell. Glucose-sensing neurons that regulate feeding in the hypothalamus share many features with the beta cell including expression of glucokinase and the ATP-sensitive potassium channel (20). The data reported herein support the prediction that malonyl-CoA may signal fuel status in hypothalamic neurons

With the escalation of obesity-related disease, mechanisms for the control of adipose balance are becoming a more crucial health issue. Taken together, the present studies provide evidence of a role for FAS in the control of feeding. As demonstrated by two distinct inhibitors of FAS, C-75 and cerulenin, this enzyme represents a potential therapeutic target for the control of appetite and body weight.

Weight Loss Agents

Weight loss agents according to this invention are agents that interfere with Neuropeptide Y expression and/or secretion and that block or reduce feeding activity. Candidate agents may be tested for their ability to reduce NPY expression by administering the agent to an animal and measuring NPY levels in the brain of the treated animal (for example as described in Example 2 for mouse brain) or by measuring NPY expression in hypothalamic cultures (see culture procedure in, e.g., Loudes, et al. (1999), “Distinct populations of hypothalamic dopaminergic neurons exhibit differential responses to brain-derived neurotrophic factor (BNDF) and neurotrophin-2 (NT3).” European Journal of Neuroscience, 11:617-624; Loudes, et al. (2000), “Brain-derived neurotrophic factor but not neurotrophin-3 enhances differentiation of somatostatin neurons in hypothalamic cultures,” Neuroendocrinology. 72(3):144-53, incorporated herein by reference). As an alternative or supplemental test, the weight loss agent may be injected intracerebroventriclularly in a test animal, and the feeding behavior of the test animal monitored (see Example 2). Preferred weight loss agents of this invention would be expected to inhibit feeding behavior.

FAS inhibitors are preferred as weight loss agents according to this invention; more preferred are FAS inhibitors that induce a reduction in expression and/or secretion of Neuropeptide Y. Therapeutic compounds are preferably compounds that inhibit FAS activity and/or raise the level of malonyl CoA without any significant (direct) effect on other cellular activities, at least at comparable concentrations. Suitable compounds for increasing malonyl CoA may be obtained as described in U.S. patent application Nos. 60/164,749, 60/164,765, and 60/164,768, incorporated herein by reference. Particularly preferred therapeutic compounds are compounds that directly reduce the activity of FAS in animal cells without any significant (direct) effect on other cellular activities, at least at comparable concentrations. As discussed above, compounds which reduce FAS activity will generally tend to increase the level of malonyl CoA.

FAS Inhibitors

A wide variety of compounds have been shown to inhibit fatty acid synthase (FAS), and selection of a suitable FAS inhibitor for use in this invention is within the skill of the ordinary worker in this art Compounds which inhibit FAS can be identified by testing the ability of a compound to inhibit fatty acid synthase activity using purified enzyme. Fatty acid synthase activity can be measured spectrophotometrically based on the oxidation of NADPH, or radioactively by measuring the incorporation of radiolabeled acetyl- or malonyl-CoA. (Dils, et al, Methods Enzymol., 35:74-83). FAS inhibitors are exemplified in U.S. Pat. No. 5.759,837, and methods of synthesizing preferred FAS inhibitors, the α-methylene-β-carboxy-γ-butyrolactones, are described in U.S. Pat. No. 5,981,575, both of which are incorporated herein by reference.

Suitable FAS inhibitors may be identified by a simple test exemplified in Example 7 of U.S. Pat. No. 5,981,575, and in U.S. Pat. No. 5,759,837, both of which are incorporated herein by reference. Generally, this test uses a tumor cell line in which an FAS inhibitor, typically cerulenin, is cytotoxic. Such cell lines include SKBR-3, ZR-75-1, and preferably HL60. Suitable FAS inhibitors will inhibit growth of such cell lines, but the cells are rescued by exogenous supply of the product of the FAS enzyme (fatty acid). When cell growth is measured in the presence and absence of exogenous fatty acid (e.g., palmitate or oleate), inhibition by specific FAS inhibitors is relieved by the fatty acid.

Alternatively, suitable FAS inhibitors can be characterized by a high therapeutic index. Inhibitors can be characterized by the concentration required to inhibit fatty acid synthesis in cell culture by 50% (IC₅₀ or ID₅₀). FAS inhibitors with high therapeutic index will inhibit fatty acid synthesis at a lower concentration (as measured by IC₅₀) than the IC₅₀ for inhibition of cell growth in the presence of exogenous fatty acid. Inhibitors whose effects on these two cellular activities show greater differences are more preferred. Preferred inhibitors of fatty acid synthesis will have IC₅₀ for fatty acid synthetic activity that is at least 1 log lower, more preferably at least 2 logs lower, and even more preferably at least 3 logs lower than the inhibitor's IC₅₀ determined for cell growth in the presence of exogenous fatty acid.

Therapy

Human therapy according to this invention will lead to decreased intracellular fat storage and a reduction in adipocyte mass. This may be expected to have the primary and/or secondary effects listed in the Table. Treatment with compounds according to this invention will lead to reduction in hepatic fat, and this in turn can lead to reduction in the rate or incidence of cirrhosis in alcoholics (see, e.g., French, 1989, Clinical Biochemistry, 22:41-9; Clements, et al., 1995, Am. J. Respir. Crit. Care Med., 151:780-784, incorporated herein by reference). Similarly, individuals with fatty livers (e.g., type II diabetics or obese persons) may benefit from administration of the agents of this invention to reduce hepatic fat (which may be detected by liver biopsy). Increased insulin responsiveness is a direct consequence of decreased adipocyte mass. Reduced adipocyte mass will reduce the risk of arterial vascular disease, stroke, etc. In patients with. elevated low density lipoproteins (LDLs), this method may be used to reduce the LDL level. Thus, the method of this invention is particularly applicable to overweight individuals, diabetics, and alcoholics. The method is generally useful as part of a program to treat obesity and complications thereof. For example, obese individuals are prone to osteoartritis, and the method of this invention may reduce the effects of the disease or delay the onset.

Table Effects of decreased intracellular fat storage and reduction in adipocyte mass

Weight loss without muscle loss
Reduction in hepatic fat
Increased insulin responsiveness (especially in Type II diabetes mellitus)
Decreased blood pressure
Decreased arterial vascular disease
Decreased susceptibility to liver injury associated with fatty change, including endotoxin mediated liver injury

The method of the present invention for inducing weight loss is applicable to animals, including vertebrates, especially mammals. Animals particularly contemplated include food animals such as poultry, swine, cattle, sheep, and other animals where reduction in fat accumulation without reduction in muscle mass may be desirable for veterinary health or economic reasons. Similarly, therapeutic compounds according to this invention, such as FAS inhibitors, may be administered according to the method of this invention to dogs, cats, horses and other animals for veterinary health reasons, particularly reasons analogous to the reasons given herein for medical therapeutic use of this invention. Dosing protocols for the compounds according to this method may be adapted to various animals from the medical procedures and the in vitro and in vivo data provided herein, in view of standard veterinary pharmacological principles. Generally, this method will not be applied to lactating animals.

Treatment according to this invention involves administering a compound according to this invention (for example, an FAS inhibitor such as an α-methylene-β-carboxy-γ-butyrolactone) to the subject of treatment. The pharmaceutical compositions containing any of the compounds of this invention may be administered by parenteral (subcutaneously, intramuscularly, intravenously, intraperitoneally, intrapleurally, intravesicularly or intrathecally), topical, oral, rectal, or nasal route, as necessitated by choice of drug and disease.

Therapeutic compounds according to this invention are preferably formulated in pharmaceutical compositions containing the compound and a pharmaceutically acceptable carrier. Therapeutic compounds may be formulated in liposomes or for administration in aerosol form. The concentrations of the active agent in pharmaceutically acceptable carriers will depend on solubilities. The dose used in a particular formulation or application will be determined by the requirements of the particular type of disease and the constraints imposed by the characteristics and capacities of the carrier materials. The pharmaceutical composition may contain other components so long as the other components do not reduce the effectiveness of the compound according to this invention so much that. the therapy is negated. Pharmaceutically acceptable carriers are well known, and one skilled in the pharmaceutical art can easily select carriers suitable for particular routes of administration (see, e.g., “Remington's Pharmaceutical Sciences,” Mack Publishing Co., Easton, Pa., 1985).

Dose and duration of therapy will depend on a variety of factors, including the therapeutic index of the drugs, disease type, patient age, patient weight, and tolerance of toxicity. Dose will generally be chosen to achieve serum concentrations from about 1 ng to about 100 μg/ml, preferably 10 ng/ml to 10 μg/ml. Preferably, initial dose levels will be selected based on their ability to achieve ambient concentrations shown to be effective in in-vitro models, such as that used to determine therapeutic index, and in-vivo models and in clinical trials, up to maximum tolerated levels. Typical doses approach 100 ng/ml in blood. Standard clinical procedure prefers that chemotherapy be tailored to the individual patient and the systemic concentration of the therapeutic agent be monitored regularly. The dose of a particular drug and duration of therapy for a particular patient can be determined by the skilled clinician using standard pharmacological approaches in view of the above factors. The response to treatment may be monitored by analysis of blood or body fluid levels of the compound according to this invention, measurement of activity if the compound or its levels in relevant tissues or monitoring disease state in the patient. The skilled clinician will adjust the dose and duration of therapy based on the response to treatment revealed by these measurements.

Preferably, the therapeutic compounds of this invention, such as FAS inhibitors, are administered based in the level necessary to control secretion of neuropeptide Y. In particular, the skilled worker is encouraged to administer FAS inhibitors to a subject so that NPY levels in the subject are at or below the level subsequent to normal feeding. Maintaining; effective NPY levels at or below the level observed following feeding will inhibit feeding behavior, and this will lead to weight loss and reduction in adipose tissue mass.

The compositions described above may be combined or used together or in coordination with another therapeutic substance. The inhibitor of fatty acid synthesis, or the synergistic combination of inhibitors, will of course be administered at a level (based on dose and duration of therapy) below the level that would kill the animal being treated. Preferably administration will be at a level that will not irreversibly injure vital organs, or will not lead to a permanent reduction in liver function, kidney function, cardiopulmonary function, gastrointestinal function, genitourinary function, integumentary function, musculoskeletal function, or neurologic function. On the other hand, administration of inhibitors at a level that kills some cells which will subsequently be regenerated (e.g., endometrial cells) is not necessarily excluded.

In addition to identifying neuropeptide Y as a key component in the pathway responsible for weight control, the present invention also provides a screening method for identifying other genes whose expression is associated with control of weight loss. Such screening can be done by comparing mRNA species expressed in tissues of an animal treated with a weight loss agent to mRNA species expressed in corresponding tissues of control animals. Procedures for obtaining total mRNA from selected tissues of treated animals are described in Example 2 for mice treated with exogenous NPY. The skilled artisan can readily provide other suitable procedures to obtain and compare mRNA expressed under treatment and control conditions, for example by adapting known techniques from the human genome project In addition, subtraction suppression hybridization, microarray or chip technology can be used to screen for differentially-expressed mRNAs (see also, Lockhart, et al. (2000), “Genomics, gene expression and DNA arrays.” Nature 405:827-836, incorporated herein by reference). In a preferred embodiment of this method, the expressed mRNA is mRNA expressed in control and treated hypothalamic tissues. Weight loss agents which are substituted α-methylene-β-carboxyl-γ-butyrolactones, such as C-75, are preferred agents for treatment of animals for comparisons according to this method. By comparing mRNA expression between treated and control animals, mRNA species associated with genes. whose expression is either up-regulated or down-regulated by the weight loss agent may be identified.

### EXAMPLES

In order to facilitate a more complete understanding of the invention, a number of Examples are provided below. However, the scope of the invention is not limited to specific embodiments disclosed in these Examples, which are for purposes of illustration only.

### Example 1

### Inhibitors of FAS and Fatty Acid Synthesis

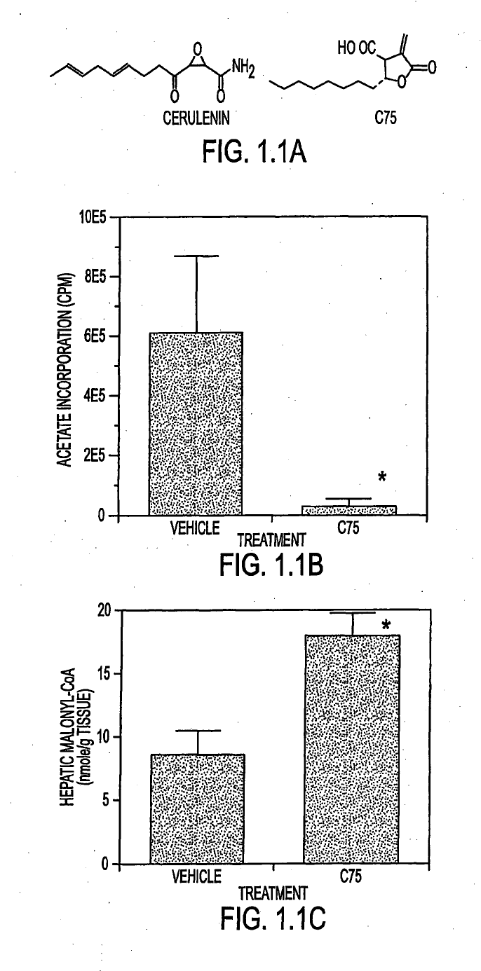

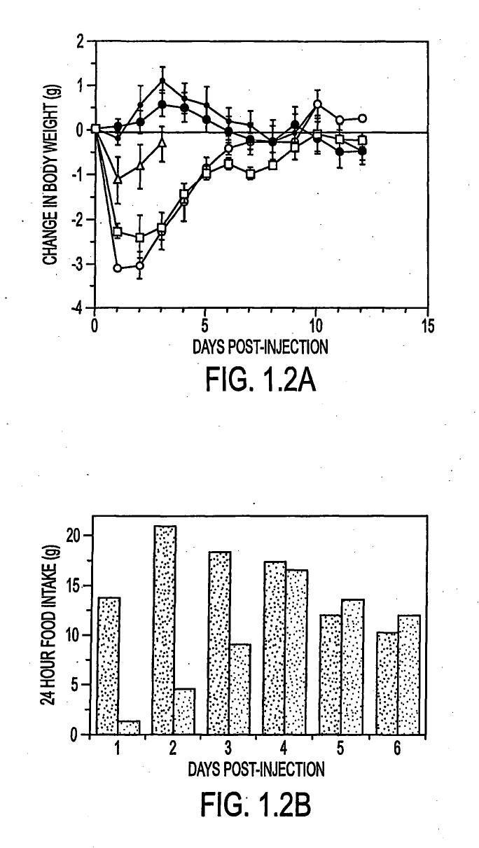

FIG. 1.1 (Panel A) shows the chemical structures of C-75 and Cerulenin. The inhibitory effects of these compounds were demonstrated on BALB/c mice.

Female BALB/c mice were treated with 0.6 mg of C-75 in 200 μl RPMI, or vehicle control IP (3 per group). After 3 hours, the animals were killed and approximately 5 mg of adipose tissue was labeled with [U-¹⁴C]-acetate, lipids were extracted and counted, [A. Rashid et al., Am. J. Pathol. 150 (1997)]. The results are shown in FIG. 1.1 (Panel B). C-75 markedly inhibited adipose fatty acid synthesis compared to vehicle control. Values represent mean +/− SEM (* P<05).

Male BALB/c mice (4 per group) were given 2 g/kg dextrose by oral gavage. After 15 min mice were injected IP with 20 mg/kg C-75 or RPMI vehicle. One hour post-treatment, livers were rapidly removed, frozen and pulverized in liquid nitrogen, HClO4 extracted and assayed for malonyl-CoA [J. D. McGarry, M. J. Stark, D. W. Foster., J. Biol.Chem 253, 8291 (1978)]. The results are shown in FIG. 1.1 (Panel C). Intraperitoneal injection of mice with C-75 leads to a 95% reduction in ¹⁴C-acetate incorporation into fatty acids and to a 110% increase in the level of hepatic malonyl-CoA, the principal substrate of FAS. Experiments described in panels B and C were repeated twice.

### Example 1A

### Effect of C-75 on Body Weight and Food Intake in Mice

The effect of C-75 treatment on feeding behavior and body weight in mice is both rapid and dramatic. A single treatment leads to the loss of as much as 20% of total body weight within 24 hours (FIG. 1.2A). This weight loss occurs in a dose dependent manner and persists for a duration that increases with dose. In all cases, treated animals recover lost body weight after the effect of the drug has dissipated, arguing against induction of a persistent wasting. The treatment is well tolerated by the mice, the only evident effect being excessive weight loss. Histological analysis of tissues from treated mice revealed no indication of adverse pathology (not shown).

Male BALB/c mice 19-22 g were weighed, treated by a single intra peritoneal (I.P.) injection and housed in metabolic cages. Body weight (FIG. 1.2A) and food intake (FIG. 1.2B) were monitored at 24 hour intervals. FIG. 1.2A shows mean change from initial body weight in mice treated with 7.5 (Δ), 15 (ο) or 30 (□)mg/kg of C-75 or RPMI vehicle (•) is expressed +/− SEM. FIG. 1.2B shows total food intake for mice treated with RPMI vehicle (black bars) or 15 mg/kg C-75 (grey bars) per day following treatment.

Inhibitors of fatty acid synthesis would be expected to prevent triglyceride accumulation due to inhibition of de novo fatty acid synthesis and impact body weight in this manner. Indeed, C-75 markedly reduces cytoplasmic triglyceride accumulation by 3T3-L1 adipocytes in cell culture (not shown). However, the dramatic C-75-induced weight loss cannot be accounted for by a blockade of fatty acid/triglyceride biosynthesis. Rather, the weight loss observed in response to C-75 treatment results primarily from an inhibition of feeding. The loss of adipose mass was accompanied by a reduction of lean body mass typical of that observed in fasting. Administration of 15 mg/kg body weight led to a greater than 90% reduction in food intake over the first 24 hours (FIG. 1.2B). Feeding behavior then returned to normal progressively over a 48-72 hour period as the drug effect dissipated. The role of feeding inhibition in C-75 induced weight loss was confirmed by studies in which forced feeding of the drug treated animals largely reversed the observed weight loss.

In concert with the feeding inhibition, there was a modest reduction in water intake, mirrored by a similar reduction in urinary output (not shown). Rather than a direct inhibition of water intake, this is consistent with a change in osmotic balance resulting from decreased intake of salts and other solutes in the diet. However, it is possible some component of the observed weight loss is due to water.

### Example 2

### Regulation of Feeding by C-75 in Fed and Fasted States: Role of NPY

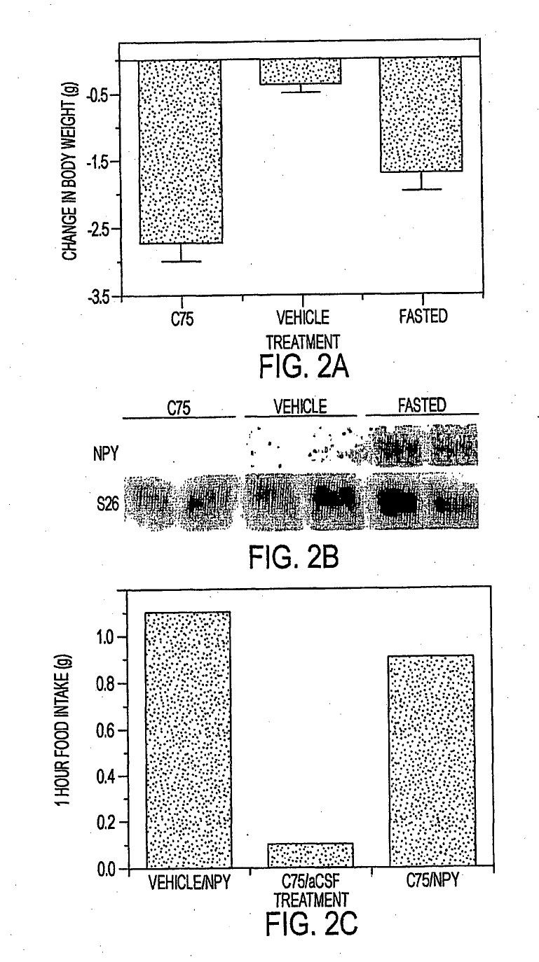

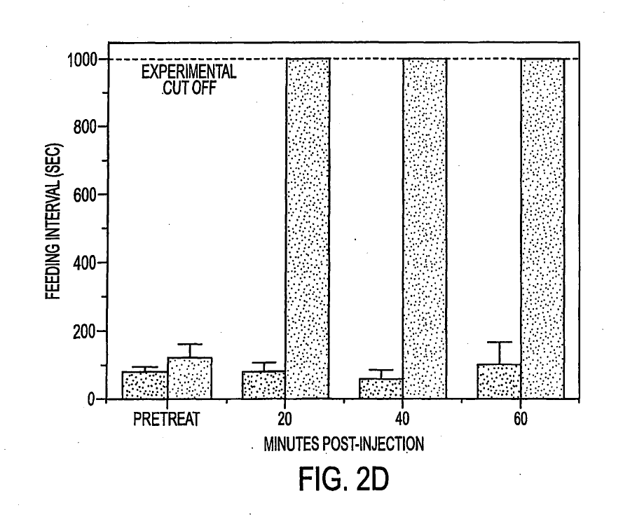

To determine whether the weight loss is attributable entirely to suppression of feeding, treatment with a dose of C-75 that completely suppresses feeding was compared with fasting. Both fasting and C-75 led to significant weight loss relative to control; however, in many experiments the C-75 treated mice lost more weight than did the fasted animals (FIG. 2A). The normal response to fasting is to reduce energy utilization to limit depletion of energy stores (Loftus, 1999). If C-75 treatment results in a “perceived fed state”, it may allow maintenance of a normal metabolic rate as well as inhibition of feeding.

Male BALB/c mice 19-21 g were preweighed and treated with vehicle or 30 mg/kg C-75 and allowed free access to food, or were denied all access to food (fasted). After 24 hours, mice were weighed. Change from initial body weight is shown in FIG. 2A, expressed as mean +/− SEM (n=7). C-75 treated mice lost 45% more weight than did the fasted animals.

The control of body weight is integrated in the hypothalamus by a coordinated group of neuropeptides that monitor adiposity and feeding status and regulate feeding and energy utilization. A central regulator in this process is neuropeptide Y (NPY) (loftus, 1999, Sem. Cell. Dev. Biol., 10:11). In the arcuate nucleus, the level of NPY increases in the fasted state (Schwartz, et al., 1998, Endocrinology, 139:2629), acting as a potent stimulus of feeding (O'Shea, et al., 1997, Endocrinology, 138:196-202). To ascertain whether C-75 might alter NPY regulation in the hypothalamus, the expression of NPY was examined by northern blot analysis of hypothalamic tissue microdissected from the brains of the fed, fasted and C-75-treated mice shown in FIG. 2A.

The hypothalamic region was microdissected from the brains of mice in FIG. 2A and total RNA was isolated. RNA was subjected to northern blot analysis using random primed probes (Feinberg, et al., 1983, Anal. Biochem., 132:6) for NPY and S26 (as a loading control). Tissue was extracted for total RNA as described, P. Chomczynski and N. Sacchi, Anal. Biochem. 162, 156 (1987). 15 μg of total RNA was subjected to Northern blot analysis as described, T. Brown, K. Mackey, in Current Protocols in Molecular Biology, F. Ausubel, et al., Eds. (John Wiley and Sons, New York, 1997) pp. 4.9.1-4.9.16. As expected, fasting markedly up-regulated NPY mRNA expression (FIG. 2B). However, the level of hypothalamic NPY mRNA in C-75-treated mice was even lower than that of the fed controls, although they had not eaten and represented the fasted state. This suggests that C-75 inhibits feeding, at least in part, by blocking the prophagic NPY signal.

To confirm this finding, the capacity of NPY to reverse C-75-induced inhibition of feeding was examined. Mice were pretreated with 30 mg/kg of C-75 by I.P. injection. After 4 hours, mice were anaesthetized by inhaled metofane and given a direct intracerebroventricular injection of 500 ng NPY (2.5 μl total volume) or artificial CSF vehicle. Mice were placed into metabolic cages and observed for feeding behavior and monitored for food intake over 18 hours. The results are shown in FIG. 2C. Total food intake within one hour by C-75/NPY treated mice was similar to that by mice treated with NPY alone and was 9 times greater than that by C-75-treated mice.

Intracerebroventricular (ICV) injection of 500 ng of NPY into mice pretreated with either vehicle or C-75 rapidly led to voracious feeding, while ICV injection of vehicle had no effect on feeding. Although the feeding effects of this dose of NPY had completely subsided in less than an hour, it was sufficient to substantially elevate the total food intake in C-75-treated mice (FIG. 2C). These results confirm both that the feeding control pathways downstream of NPY are intact in C-75-treated mice, and that C-75 acts upstream of NPY release, as anticipated from the northern blot analysis.

The effect of C-75 on feeding was also examined with fasted mice which exhibit up-regulated NPY levels, and feed voraciously. Mice were fasted for 24 hours to induce voracious feeding. Initial feeding interval (time in seconds between food presentation and initiation of feeding) was measured in naive mice (pretreat). Mice were then treated by I.P. injection of 30 mg/kg C-75 or RPMI vehicle and feeding interval determined at 20, 40 and 60 minutes post-injection. The results are shown in FIG. 2D. Observation was terminated if no feeding was initiated within 1000 sec (experimental cut off). Times represent mean +/− SEM, (n=4).

Prior to treatment, all animals fed ravenously within 3 minutes of being offered food. However, within 20 minutes of C-75 treatment, the mice lost all interest in feeding, while vehicle treated mice continued to initiate feeding within 3 minutes of food presentation (FIG. 2D). The fact that these animals had already up-regulated their NPY message levels indicates that C-75 must have additional. actions, either on NPY release, or on other regulators of feeding behavior.

### Example 3

### Leptin Independence of C-75 Action and Treatment of ob/ob Mice

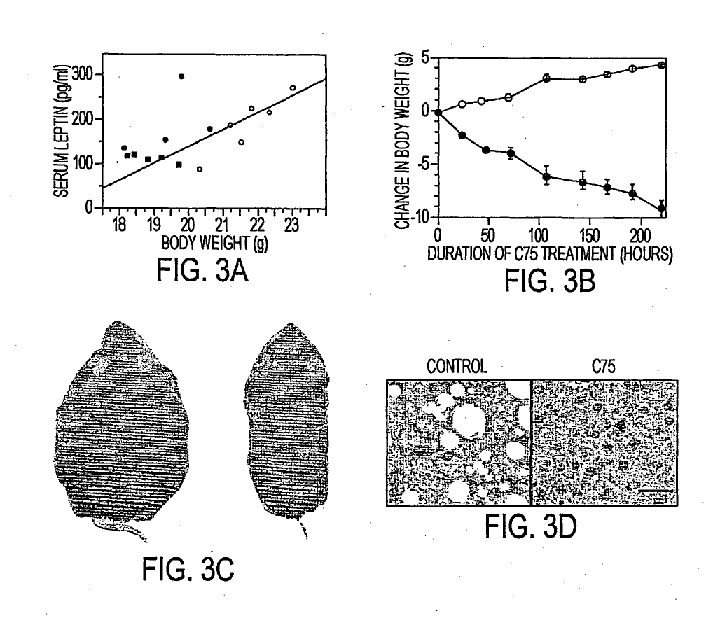

One of the primary signals modulating NPY function in feeding control is leptin. This hormone is elevated in the fed state and inhibits NPY production and feeding (Schwartz, et al., 1996, Diabetes, 45:531) in a manner similar to that observed with C-75 treatment. Leptin was an attractive candidate as its primary site of production, white adipose tissue (Zhang, et al., 1994, Nature, 372:425), is a site of fatty acid synthesis and expresses high levels of FAS. To test for increased leptin release as the signal mediating C-75 regulation of NPY, serum leptin levels were assessed in fed (end of light cycle) fasted and C-75-treated mice. BALB/c mice treated with RPMI vehicle (ο) or 30 mg/kg C-75 (▪) I.P. and free fed, or fasted (•) for 24 hours were weighed, decapitated and exsanguinated. Serum leptin levels were determined using a Quantikine murine leptin ELISA. (R&D Systems) and plotted against total body weight (FIG. 3A). Rather than elevation, a reduction in leptin levels was observed. This reduction correlates with the reduction in body weight, presumably body fat, resulting from C-75 treatment FIG. 3A). This is consistent with the normal regulation of leptin levels during weight loss (Boden, et al., 1996, J. Clin. Endocrinol. Metab., 81:3419) and indicates that leptin does not mediate the C-75 signal. Northern blot analysis of leptin message levels in white adipose tissue from the same animals (performed as described above) supports this observation (data not shown).

A leptin independent mechanism suggested that C-75 should be effective in reducing the obesity of ob/ob mice which do not express functional leptin (Schwartz, et al., 1996). This was confirmed over a two week course of treatment which led to a substantial reduction in the body weight of C-75-treated animals while vehicle treated mice continued to gain weight (FIG. 3B). Male ob/ob (C57BL/6OlaHsd-Lep^{ob}, Harlan) mice were treated with RPMI vehicle (ο) or 22 mg/kg C-75 (•) I.P. every third day and body weight monitored change in body weight is displayed as mean +/− SEM. The magnitude of this effect is readily evident by inspection of representative C-75 and control treated ob/ob mice. (See FIG. 3C, which shows representative vehicle and C-75 treated mice from FIG. 3B at the termination of treatment (14 days)).

C-75 treatment not only led to weight loss, but also corrected many of the pathological consequences that result from the extreme obesity of ob/ob mice. Liver samples from vehicle and C-75 treated mice (from FIG. 3B) were fixed in formalin and paraffin embedded. Tissue sections (4 μm) were stained with hematoxylin and eosin. Histological examination of the liver from C-75 treated animals showed a marked reduction in the hepatomegaly and fatty liver observed in control ob/ob mice (FIG. 3D, scale bar=50 μ). Analysis of white adipose tissue demonstrated a dramatic reduction in mean adipocyte size (not shown). There was no evidence of histological. abnormality resulting from chronic treatment of the animals even in these primary tissues of fatty acid synthesis. The observation that C-75 acts through a leptin independent mechanism is particularly promising in that the majority of obese individuals appear to be relatively resistant to leptin's effects (Caro, et al., 1996, Lancet, 348:159).

### Example 4

### C-75 Treatment Corrects Hyperglycemia in ob/ob Mice

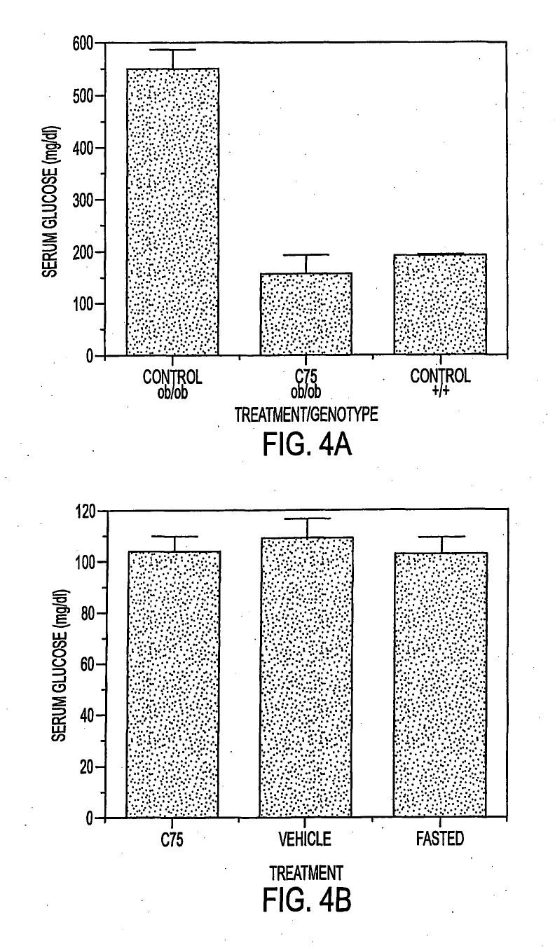

In addition to obesity, ob/ob mice also develop overt diabetes with significant elevation of blood glucose. C-75 corrected the hyperglycemia observed in vehicle treated mice with a nearly 3-fold reduction in mean serum glucose (FIG. 4A). Male ob/ob mice (n=3) were treated with C-75 or vehicle for 2 weeks (FIGS. 3B and C) and compared with age matched, untreated c57BL/6j mice (+/+) 24 hour IP treatment of wild-type mice had no effect on serum glucose beyond that attributable to fasting. The normalization of blood glucose occurred from the profound weight loss in the ob/ob mice as acute treatment of normal mice with C-75 had no effect on serum other than that resulting from inhibition of feeding (FIG. 4B). Male BALB/c mice (n=7) were fasted for 24 hours or injected IP with 30 mg/kg C-75 or RPMI vehicle and allowed free access to food for 24 hours. In both cases, serum was collected at death and assayed for glucose: Ref Lab™ GLU (Medical Analysis Systems, Inc., Camarillo, Calif.). These data highlight the importance of C-75's independence from leptin, since over 75% of obese humans appear to be resistant to leptin's effects. Both panels are representative of 2 experiments.

### Example 5

### Regulation of Feeding by Malonyl CoA

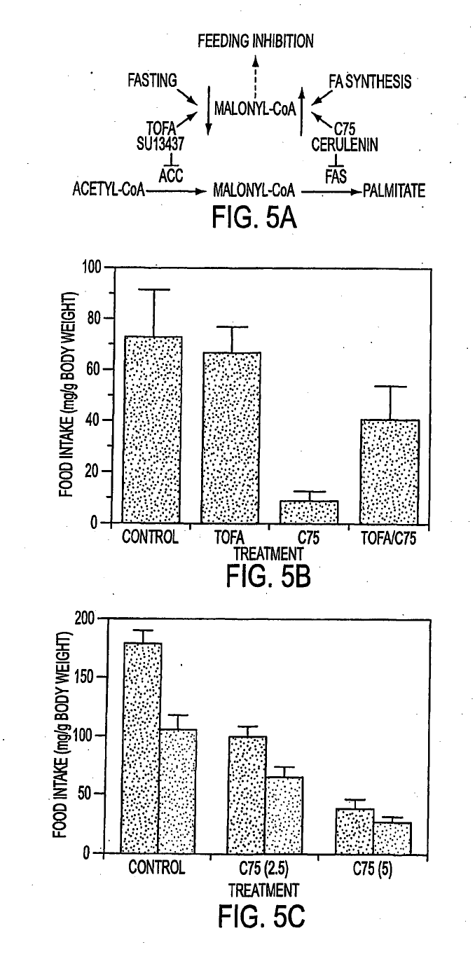

FIG. 5A shows a model of feeding regulation by inhibitors of FAS via malonyl-CoA. This model predicts that feeding inhibition by FAS inhibitors should be attenuated by inhibitors of ACC's. To test this, mice were pretreated with the ACC inhibitor TOFA or vehicle by ICV injection and examined the ability of C-75, administered IP, to inhibit feeding. BALB/c mice were anesthetized with metofane and injected ICV with 2 μg of TOFA or DMSO vehicle. After 2 hours recovery, mice were injected IP with 15 mg C-75/kg or RPMI vehicle and monitored for total food intake over 2 hours. TOFA largely restored food intake in C-75-treated mice (FIG. 5B), supporting the hypothesis that malonyl-CoA mediates feeding inhibition. In addition, mice were anesthetized and injected ICV with 2 μl of RPMI or C-75 at 2.5 or 5 μg/μl and food intake monitored over 2 (shaded) and 4 (solid) hours. The efficacy of centrally administered TOFA argues for a central (CNS) mechanism of action. ICV administration of C-75 inhibited feeding by 82% (FIG. 5C), supporting the central target action of C-75. FIG. 5 (B) and (C) combine results from 3 experiments with N=3 for each (9 total).

### Example 6

### Immunohistochemical Localization of Malonyl CoA Metabolism

Antibodies specific for the enzymes fatty acid synthase, acetyl-CoA carboxylase alpha isoform, and malonyl-CoA decarboxylase may be used to detect the presence of the respective enzymes in neural tissue. Fatty acid synthase, acetyl-CoA carboxylase alpha isoform, and malonyl-CoA decarboxylase all co-localize to the arcuate nucleus of the hypothalamus in mice by standard methods of immunohistochemical detection using these antibodies. The arcuate nucleus is important in appetite control in the hypothalamus.

For purposes of clarity of understanding, the foregoing invention has been described in some detail by way of illustration and example in conjunction with specific embodiments, although other aspects, advantages and modifications will be apparent to those skilled in the art to which the invention pertains. The foregoing description and examples are intended to illustrate, but not limit the scope of the invention. Modifications of the above-described modes for carrying out the invention that are apparent to persons of skill in clinical medicine, physiology, pharmacology, and/or related fields are intended to be within the scope of the invention, which is limited only by the appended claims.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

1. A method for inducing weight loss in an animal, comprising administering to the animal a compound which reduces the expression and/or secretion of neuropeptide Y (NPY).

2. The method of claim 1, wherein administration of the compound increases malonyl CoA levels in the animal.

3. The method of claim 1, wherein the compound is an inhibitor of fatty acid synthase (FAS) and is administered in an amount sufficient to reduce the expression and/or secretion of NPY.

4. The method of claim 1, wherein the compound is a substituted α-methylene-β-carboxyl-γ-butyrolactone.

5. The method of claim 1, wherein the compound is an inhibitor of malonyl Coenzyme A decarboxylase (MCD).

6. The method of any one of claims 1 to 5, wherein the compound is administered in an amount sufficient to reduce expression of NPY at least to the level observed in fed animals.

7. The method of any one of claims 1 to 5, wherein expression and/or secretion of NPY is reduced in cells which express FAS.

8. The method of claim 1, wherein administration of the compound inhibits feeding behavior in the animal.

9. The method of claim 1, wherein the animal is deficient in expression of leptin or the animal is resistant to leptin.

10. A screening method to aid in identifying weight loss agents comprising administering a candidate compound to an animal or a hypothalamic culture; and monitoring expression or secretion of neuropeptide Y.

11. The screening method according to claim 10, wherein the treated animal is monitored for reduced frequency or intensity of feeding.

12. The method according to claim 10, wherein the candidate compound is administered to the animal by injection.

13. The method according to claim 12, wherein the compound is administered intraperitoneally or intracerebroventricularly.

14. The method according to any one of claims 10-13, wherein the candidate compound is an inhibitor of the enzyme fatty acid synthase.

15. A screening method for identifying genes whose expression is associated with control of weight loss comprising
administering a weight loss agent to an animal; and
comparing expressed mRNA species in the animal treated with the weight loss agent to expressed mRNA species in control animals,
wherein mRNA species expressed differentially are associated with control of weight loss.

16. The method of claim 15, wherein the weight loss agent is an substituted α-methylene-β-carboxyl-γ-butyrolactone, such as C-75.

17. The method of claim 15, wherein comparison of expressed mRNA species is limited to hypothalamic mRNA.
